(19) United States
(12) Patent Application Publication
(10) Pub. No.: US 2024/0156622 A1
(43) Pub. Date: May 16, 2024

(54) BALLOON EXPANDABLE STENT PROSTHESES AND BALLOON EXPANDABLE BRANCHING STENT PROSTHESES

(71) Applicant: Merit Medical Systems, Inc., South Jordan, UT (US)

(72) Inventors: John Hall, Bountiful, UT (US); Wayne Mower, Bountiful, UT (US); Christopher Cindrich, Highland, UT (US); Lindsay Koren, South Jordan, UT (US); Adam Wilhite, Herriman, UT (US)

(21) Appl. No.: 18/508,539

(22) Filed: Nov. 14, 2023

Related U.S. Application Data
(60) Provisional application No. 63/383,881, filed on Nov. 15, 2022.

(51) Int. Cl. A61F 2/82; A61F 2/958

(52) U.S. Cl. CPC A61F 2/82; A61F 2/958

(57) ABSTRACT

A balloon expandable branching stent prosthesis are discussed herein. The balloon expandable branching stent prosthesis includes a trunk portion including a trunk portion stent frame, a first branching portion including a first branching portion stent frame, a second branching portion including a second branching portion stent frame, and a cover coupling the trunk portion stent frame, the first branching stent frame, and the second branching stent frame together forming a bifurcated stent, wherein the trunk portion stent frame, the first branching portion stent frame, and the second branching portion stent frame are spaced apart from each other with the cover disposed therebetween. A crotch portion of the balloon expandable branching stent prosthesis is disposed between the trunk portion, the first branching portion, and the second branching portion and the crotch portion is reinforced to prevent radial infolding of the cover.

### RELATED APPLICATIONS

This application claims priority to U.S. Provisional Application No. 63/383,881, filed on Nov. 15, 2022 and titled, “BALLOON EXPANDABLE STENT PROSTHESES AND BALLOON EXPANDABLE BRANCHING STENT PROSTHESES,” which is hereby incorporated by reference in its entirety.

### TECHNICAL FIELD

The present disclosure relates generally to the field of balloon expandable stent prostheses. More particularly, some embodiments relate to bifurcated expandable stent prostheses.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A illustrates a balloon expandable branching stent prosthesis according to one embodiment of the present disclosure.

FIG. 1B illustrates a balloon expandable branching stent prosthesis according to one embodiment of the present disclosure.

FIG. 2A illustrates the balloon expandable branching stent prosthesis of FIG. 1A implanted in a patient.

FIG. 2B illustrates the balloon expandable branching stent prosthesis of FIG. 1B implanted in a patient.

FIG. 3A illustrates an unassembled configuration of a stent frame according to one embodiment of the present disclosure.

FIG. 3B illustrates a side view of the stent frame of FIG. 3A in an assembled configuration forming a tubular stent frame.

FIG. 3C illustrates a perspective view of the stent frame of FIG. 3A in an assembled configuration forming a tubular stent frame.

FIG. 4A illustrates an unassembled configuration of a stent frame according to one embodiment of the present disclosure.

FIG. 4B illustrates a side view of the stent frame of FIG. 4A in an assembled configuration forming a tubular stent frame.

FIG. 4C illustrates a perspective view of the stent frame of FIG. 4A in an assembled configuration forming a tubular stent frame.

FIG. 5A illustrates a side view of a stent frame in an assembled configuration forming a tubular stent frame according to one embodiment of the present disclosure.

FIG. 5B illustrates a perspective view of the stent frame of FIG. 5A.

FIG. 6A illustrates an unassembled configuration of a stent frame according to one embodiment of the present disclosure with a plurality of thickness zones.

FIG. 6B illustrates an unassembled configuration of a stent frame according to one embodiment of the present disclosure with a plurality of thickness zones.

FIG. 7A illustrates a cross-sectional view of the stent frame of FIG. 6B in a compressed configuration.

FIG. 7B illustrates a cross-sectional view of the stent frame of FIG. 6B in a partially expanded configuration.

FIG. 7C illustrates a cross-sectional view of the stent frame of FIG. 6B in an expanded configuration.

FIG. 8A illustrates a perspective view of a stent frame with a flared end according to one embodiment of the present disclosure.

FIG. 8B illustrates a balloon expandable branching stent prosthesis with the flared stent frame of FIG. 8A in a trunk portion of the balloon expandable branching stent prosthesis implanted in a patient according to one embodiment of the present disclosure.

FIG. 9A illustrates a balloon expandable branching stent prosthesis with a reinforced crotch portion according to one embodiment of the present disclosure.

FIG. 9B illustrates a balloon expandable branching stent prosthesis with a reinforced crotch portion according to one embodiment of the present disclosure.

FIG. 10 illustrates a balloon expandable branching stent prosthesis with a strap the extend from a first side of a trunk stent frame under the crotch portion to a second side of the trunk stent frame to reinforce a crotch portion according to one embodiment of the present disclosure.

FIG. 11 illustrates a flowchart of manufacturing a stent prosthesis according to one embodiment of the present disclosure.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

Embodiments may be understood by reference to the drawings, wherein like parts are designated by like numerals throughout. It will be understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

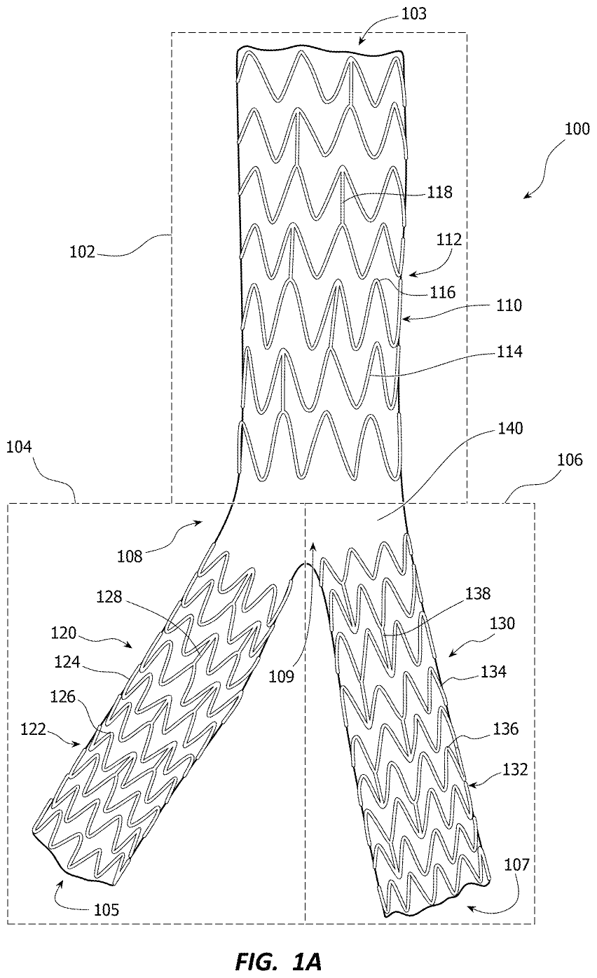

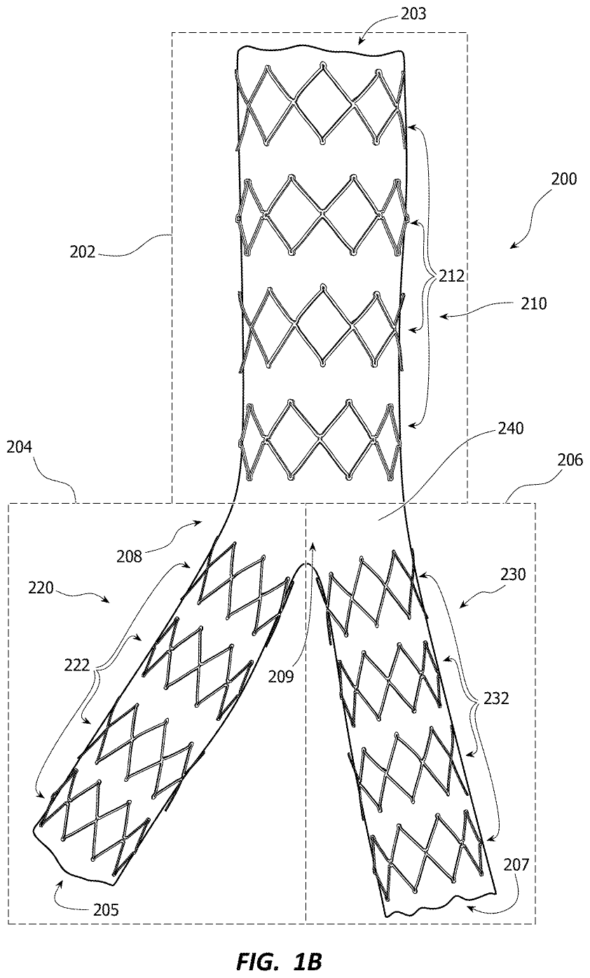

FIG. 1A illustrates a perspective view of a balloon expandable branching stent prosthesis 100, according to embodiments herein. The balloon expandable branching stent prosthesis 100 of FIG. 1A is illustrated in a deployed (expanded) configuration. The balloon expandable branching stent prosthesis 100 is configured to be expanded to the deployed configuration from an unexpanded/undeployed configuration via inflation of one or more balloons in the lumen of the balloon expandable branching stent prosthesis 100.

The balloon expandable branching stent prosthesis 100 may include a trunk portion 102, a first branching portion 104, and a second branching portion 106. The trunk portion 102, the first branching portion 104, and the second branching portion 106 join together at a crotch portion 108. The trunk portion 102 comprises a lumen 103 that extends from a first end (e.g., proximal end opposite the crotch portion 108) to a second end (e.g., distal end near the crotch portion 108) of the trunk portion 102, the first branching portion 104 comprises a lumen 105 that extends from a first end (e.g., proximal end near the crotch portion 108) to a second end (e.g., distal end opposite the crotch portion 108) of the first branching portion 104, and the second branching portion 106 comprises a lumen 107 that extends from a first end (e.g., proximal end near the crotch portion 108) to the second end (e.g., distal end opposite the crotch portion 108) of the second branching portion 106. Each lumen 103, 105, 107 are in fluid communication with each other and converge at a branch point 109.

In the illustrated embodiment, the balloon expandable branching stent prosthesis 100 is a bifurcated stent prosthesis. In other words, the bifurcated stent prosthesis branches into two branches or arms. In some embodiments, the balloon expandable branching stent prosthesis 100 may comprise multiple branches, such as three, four, five, etc.

In the illustrated embodiment, the first branching portion 104 and the second branching portion 106 of the balloon expandable branching stent prosthesis 100 may have a similar diameter. The trunk portion 102 of the balloon expandable branching stent prosthesis 100 may have a greater diameter of the first branching portion 104 and the second branching portion 106. In some embodiments, the trunk portion 102 may have a similar diameter to the first branching portion 104 and the second branching portion 106. In some embodiments, the trunk portion 102 may have a smaller diameter than the first branching portion 104 and the second branching portion 106. In some embodiments the cross-sectional area of the trunk portion 102 may be similar to the sum of the cross-sectional areas of the first branching portion 104 and the second branching portion 106.

In the illustrated embodiment, the balloon expandable branching stent prosthesis 100 is partially composed of three separate stent frames, 110, 120, 130 and a cover 140. A first stent frame 110 is disposed in the trunk portion 102, the second stent frame 120 is disposed in the first branching portion 104, and the third stent frame 130 is disposed in a second branching portion 106. The stent frames 110, 120, 130 may also be referred to as the trunk stent frame 110, the first branching stent frame 120, and the second branching stent frame 130. The cover 140 is configured to couple the stent frame 110, 120, 130 together to form the balloon expandable branching stent prosthesis 100.

As discussed above, the crotch portion 108 of the balloon expandable branching stent prosthesis 100 is when the three branches of the balloon expandable branching stent prosthesis 100 conjoin. The crotch portion 108 acts as a transition zone between the three separate stent frame 110, 120, 130. Specifically, the first stent frame 110 is separated from the second stent frame 120 and the third stent frame 130 by a predetermined distances but coupled together by the cover 140, the second stent frame 120 is separated from the first stent frame 110 and the third stent frame 130 by a predetermined distances but coupled together by the cover 140, and the third stent frame 130 is separated from the first stent frame 110 and the second stent frame 120 by a predetermined distances but coupled together by the cover 140.

Each stent frame 110, 120, 130 are configured to be expandable to the illustrated deployed position from an unexpanded/undeployed configuration (e.g., via the inflation of one or more balloons from within the balloon expandable branching stent prosthesis 100) after the balloon expandable branching stent prosthesis 100 is delivered to a desired location.

Each stent frame 110, 120, 130 may comprise a frame 112, 122, 132 configured to provide a chronic radial outwardly directed force and a resistance to a radial compression when disposed in a body lumen of a patient. In the illustrated embodiment, the frame 112, 122, 132 may comprise a plurality of arms 114, 124, 134 that form a plurality of apexes 116, 126, 128. Adjacent apexes 116, 126, 136 may be coupled together by struts 118, 128, 138. The frame 112, 122, 132 may be formed of any suitable material, such as stainless steel, tantalum, tantalum alloys, platinum alloys, niobium alloys, cobalt alloys, other metallic alloys, polymers, and the like. The stent frames 110, 120, 130 are not limited to the illustrated embodiment, but may have variety of different patterns, such as a zig-zag pattern, a wave pattern, or any other suitable pattern. Each stent frame 110, 120, 130 may be pre-formed or formed corresponding to a tubular body. The material, pattern, and diameter of each stent frame 110, 120, 130 may be configured to provide a chronic radial outwardly directed force and a resistance to a radial inwardly directed force.

In some embodiments, the frame 112, 122, 132 may consist of a single continuous wire forming a plurality of helixes that wrap around forming the lumens of the stent frame 110, 120, 130.

The cover 140 may be formed of a variety of materials and/or layers of materials, including biocompatible materials that are resistant to passage of fluid through the cover 140. For example, the cover 140 may be formed of polyethylene terephthalate, polyurethane, silicone rubber, nylon, fluoropolymer, polyester, etc. A thickness of the wall may range from about 0.07 mm to about 0.5 mm.

In some embodiments, the cover 140 may comprise a plurality layers. In some embodiments, the cover 140 may comprise an outer layer, a tie layer, and an inner layer. In some embodiments the tie layer may be configured to promote bonding between the outer layer and the inner layer. In other embodiments the tie layer may further be configured to provide certain properties to the balloon expandable branching stent prosthesis 100 as a whole, such as stiffness or tensile strength. In some embodiments, the inner layer may be rotational spun polytetrafluoroethylene (PTFE), the tie layer may be fluorinated ethylene propylene (FEP), and the outer layer may be expanded PTFE (ePTFE). In some embodiments, the tie layer may be elastic material, a thermoplastic material, silicone, and the like. However, the present disclosure is not so limited and other configurations of the layers are also within the scope of the present disclosure.

Additionally, in embodiments where both the inner layer and the outer layer are porous in nature, the tie layer may be configured to create an impermeable layer between the two porous layers. In such embodiments the balloon expandable branching stent prosthesis 100 may permit tissue ingrowth, tissue attachment and/or healing on both the inner and outer surfaces of the stent while still preventing tissue outside of the stent from growing into the lumen and occluding the lumen. Thus, the tie layer may be configured to create a mid-layer portion of a construct and the tie layer is tissue or cell impermeable to inhibit tissue or cellular ingrowth into the layer or to be impervious to tissue migration into or through the layer or to substantially inhibit tissue migration.

In certain embodiments, a wall of the cover 140 may be impermeable to tissue or cellular ingrowth into and/or tissue cell migration through the wall, for example, to prevent or discourage stenosis of the cover 140. Additionally or alternatively, in some embodiments, the wall of the cover 140 can be impermeable to fluid such that fluid is prevented from leaking from the inside of the balloon expandable branching stent prosthesis 100 to the exterior of the balloon expandable branching stent prosthesis 100 and into surrounding tissue. In some embodiments an interior surface of the wall may include serially deposited fibers of polytetrafluoroethylene (PTFE) to resist fibrin deposition and platelet adhesion on the surfaces.

Note that in embodiments herein, stent prostheses are illustrated as comprising covers or tubular bodies (such as the cover 140 of FIG. 1A). However, it will be understood that expandable branching stent prostheses using a wire scaffold, framework, or stent without a cover or tubular body coupled thereto fall within the scope of the disclosure.

FIG. 1B depicts an embodiment of a balloon expandable branching stent prosthesis 200 that resembles the balloon expandable branching stent prosthesis 100 described above in certain respects. Accordingly, like features are designated with like reference numerals, with the leading digits incremented to “2.” For example, the embodiment depicted in FIG. 1B includes a trunk portion 202 that may, in some respects, resemble the trunk portion 102 of FIG. 1A. Relevant disclosure set forth above regarding similarly identified features thus may not be repeated hereafter. Moreover, specific features of the balloon expandable branching stent prosthesis 100 and related components shown in FIG. 1A may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the balloon expandable branching stent prosthesis 200 and related components depicted in FIG. 1B. Any suitable combination of the features, and variations of the same, described with respect to the balloon expandable branching stent prosthesis 100 and related components illustrated in FIG. 1A can be employed with the balloon expandable branching stent prosthesis 200 and related components of FIG. 1B, and vice versa. This pattern of disclosure applies equally to further embodiments depicted in subsequent figures and described hereafter, wherein the leading digits may be further incremented.

FIG. 1B illustrates a perspective view of the balloon expandable branching stent prosthesis 200, according to embodiments herein. The balloon expandable branching stent prosthesis 200 may include the trunk portion 202, a first branching portion 204, and a second branching portion 206. The trunk portion 202, the first branching portion 204, and the second branching portion 206 join together at a crotch portion 208. The trunk portion 202 comprises a lumen 203 that extends from a first end to a second end of the trunk portion 202, the first branching portion 204 comprises a lumen 205 that extends from a first end to a second end of the first branching portion 204, and the second branching portion 206 comprises a lumen 207 that extends from a first end to the second end of the second branching portion 206. Each lumen 203, 205, 207 are in fluid communication with each other and converge at a branch point 209.

In the illustrated embodiment, the balloon expandable branching stent prosthesis 200 is partially composed of three separate stent frames, 210, 220, 230 and a cover 240. A first stent frame 210 is disposed in the trunk portion 202, the second stent frame 220 is disposed in the first branching portion 204, and the third stent frame 230 is disposed in a second branching portion 206. The cover 240 is configured to couple the stent frame 210, 220, 230 together to form the balloon expandable branching stent prosthesis 200.

Each stent frame 210, 220, 230 are configured to be expandable to the illustrated deployed position from an unexpanded/undeployed configuration (e.g., via the inflation of one or more balloons from within the balloon expandable branching stent prosthesis 200) after the balloon expandable branching stent prosthesis 200 is delivered to a desired location. Each stent frame 210, 220, 230 may be formed of any suitable material, such as stainless steel, tantalum, tantalum alloys, platinum alloys, niobium alloys, cobalt alloys, other metallic alloys, polymers, and the like. Each stent frame 210, 220, 230 may have a plurality of annular rings 212, 222, 232 with variety of different patterns, such as a diamond pattern, or any other suitable pattern. In the illustrated embodiment, the plurality of annular rings 212, 222, 232 comprise a plurality of diamonds forming an annular ring. In the illustrated embodiment, the plurality of annular rings 212, 222, 232 are separated from each other and are not connected. In some embodiments, the annular rings 212, 222, 232 may be coupled to together with a plurality of struts. The diamond pattern enables the annular rings 212, 222, 232 to compress and expand. In the compressed configuration, each diamond is flattened, as the annular ring expands, each diamond elongates expanding the annular ring 212, 222, 232. Each stent frame 210, 220, 230 may be pre-formed or formed corresponding to a tubular body. The material, pattern, and diameter of each stent frame 210, 220, 230 may be configured to provide a chronic radial outwardly directed force and a resistance to a radial inwardly directed force.

As illustrated herein, the balloon expandable branching stent prosthesis 100, 200 may be for deployment at a branching implant site within a body having a trunk, a first branch, and a second branch. Accordingly, the balloon expandable branching stent prosthesis 100, 200 may anatomically designed so that the trunk portion 110, 210, the first branching portion 120, 220, and the second branching portion 130, 230 for an anatomical fit at the implant site. For deployment to the balloon expandable branching stent prosthesis 100, 200 at the branching implant site, the trunk portion 110, 210 of the balloon expandable branching stent prosthesis 100, 210 is used in/at the trunk of the branching implant site, the first branching portion 120, 220 is deployed in/at the first branch of the branching implant site, and the second branching portion 130, 230 is deployed in/at the second branch of the branching implant site.

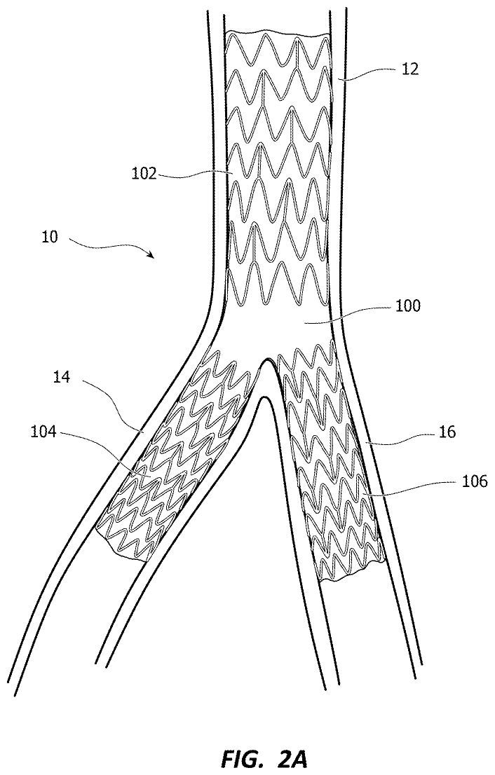

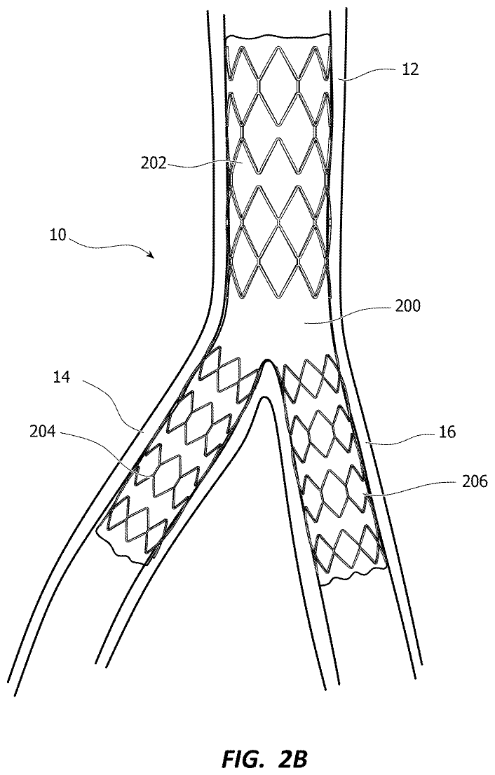

FIG. 2A illustrates the balloon expandable branching stent prosthesis 100 deployed at a branching implant site 10, according to embodiments herein. In this expanded/deployed configuration, the balloon expandable branching stent prosthesis 100 provides stenting at/through the branching implant site 10 within the anatomical system in which it is deployed.

The branching implant site 10 may be a portion of an anatomical system that includes a trunk vessel 12, a first branch vessel 14, and a second branch vessel 16 in which blood flows through. In the illustrated deployed configuration, the trunk portion 102 of the balloon expandable branching stent prosthesis 100 is deployed at/within the trunk vessel 12 of the branching implant site 10, the first branching portion 104 of the balloon expandable branching stent prosthesis 100 is deployed at/within the first branch vessel 14 of the branching implant site 10, and the second branching portion 106 of the balloon expandable branching stent prosthesis 100 is deployed at/within the second branch vessel 16 of the branching implant site 10.

In some embodiments, the balloon expandable branching stent prosthesis 100, once deployed, provides an appropriate channel for desired fluid flow through the branching implant site 10. It may be that the region around the branching implant site 10 is diseased, misshapen, and/or damaged, and that the deployment of the balloon expandable branching stent prosthesis 100 in the illustrated manner can correct and/or ameliorate attendant issues.

FIG. 2B illustrates the balloon expandable branching stent prosthesis 200 deployed at the branching implant site 10, according to embodiments herein. In this expanded/deployed configuration, the balloon expandable branching stent prosthesis 200 provides stenting at/through the branching implant site 10 within the anatomical system in which it is deployed.

The branching implant site 10 may be a portion of an anatomical system that includes a trunk vessel 12, a first branch vessel 14, and a second branch vessel 16 in which blood flows through. In the illustrated deployed configuration, the trunk portion 202 of the balloon expandable branching stent prosthesis 200 is deployed at/within the trunk vessel 12 of the branching implant site 10, the first branching portion 204 of the balloon expandable branching stent prosthesis 200 is deployed at/within the first branch vessel 14 of the branching implant site 10, and the second branching portion 206 of the balloon expandable branching stent prosthesis 200 is deployed at/within the second branch vessel 16 of the branching implant site 10.

In some embodiments, the balloon expandable branching stent prosthesis 200, once deployed, provides an appropriate channel for desired fluid flow through the branching implant site 10. It may be that the region around the branching implant site 10 is diseased, misshapen, and/or damaged, and that the deployment of the balloon expandable branching stent prosthesis 200 in the illustrated manner can correct and/or ameliorate attendant issues.

As illustrated, the branching implant site 10 of FIGS. 2A and 2B is used within an anatomical system that is a cardiovascular system. However, it should be noted that while examples herein describe (and figures herein illustrate) the deployment of expandable branching stent prostheses within cardiovascular systems, the cardiovascular anatomical context is given by way of example and not by way of limitation. It will be understood that deployment systems analogous to those described in relation to disclosure herein may be used to deploy appropriate expandable branching stent prostheses with/at branching implant sites of other anatomical systems, and that corresponding methods for using such deployment systems to deploy corresponding expandable branching stents that are analogous to those methods described herein could be used in those other anatomical contexts.

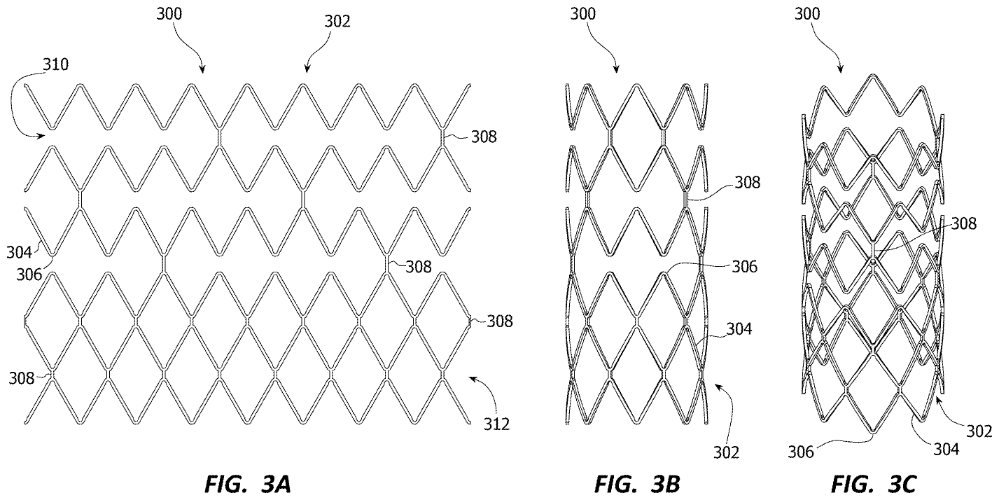

FIGS. 3A-3C illustrate a stent frame 300 according to one example of the present disclosure. The stent frame 300 may be utilize in the balloon expandable branching stent prosthesis 100. For example, the stent frame 300 may be utilized as the stent frame 110, 120, 130 in the balloon expandable branching stent prosthesis 100.

FIG. 3A illustrates an unassembled configuration of the stent frame 300. FIG. 3B illustrates a side view of the stent frame 300 in an assembled configuration forming a tubular stent frame. FIG. 3C is a perspective view of the stent frame 300 in an assembled configuration forming a tubular stent frame. In the illustrated embodiment, the stent frame 300 includes a frame 302 with a plurality of arms 304 forming apexes 306. The laterally adjacent arms 304 form V-shaped structures. Accordingly, the frame 302 forms a plurality a V-shaped structures. Vertically adjacent V-shape structures can form diamond shaped structures.

In the illustrated embodiment of the stent frame 300, the frame 302 comprises six rows of arms 304. The rows of the frame 302 may be coupled together by struts 308. The struts 308 may couple vertically adjacent apexes 306. The length of the struts 308 in the bottom three rows are shorter than the length of the struts 308 in the top three rows. Further, the struts 308 in the bottom three rows couple every other apex 306 to a vertical adjacent apex 306 and the struts 308 disposed between the first row and the second row are offset from the struts 308 between the second row and the third row. The struts 308 between rows between three, four, five, and six are offset from each other. The struts 308 in these rows may be separated by several apexes 306.

In some embodiments, the stent frame 300 may be cut from a sheet of material. For example, the stent frame 300 may be cut from a sheet a material and then formed into a tubular shape. For example, a first end 310 of the stent frame 300 may be coupled to a second end 312 of the stent frame 300 to form a tubular shape. In some embodiment, the stent frame 300 may be cut from a tube of material.

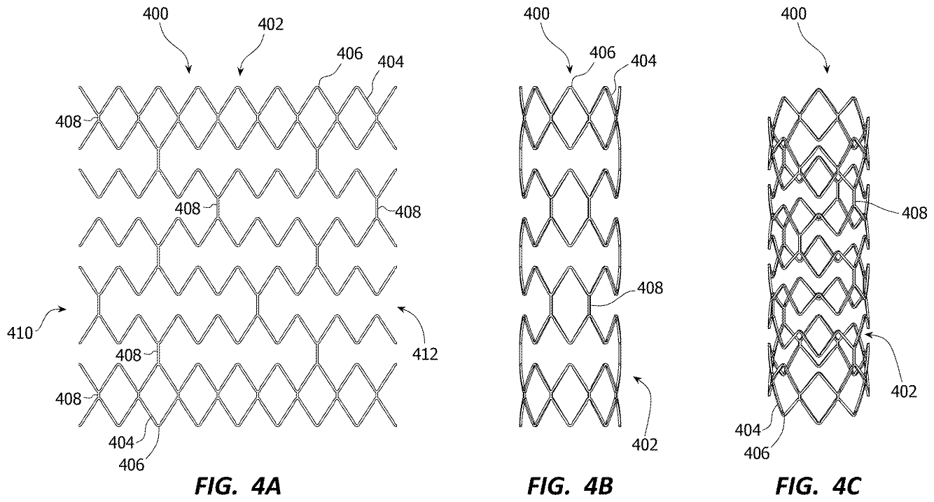

FIGS. 4A-4C illustrate a stent frame 400 according to one example of the present disclosure. The stent frame 400 may be utilize in the balloon expandable branching stent prosthesis 100. For example, the stent frame 400 may be utilized as the stent frame 110, 120, 130 in the balloon expandable branching stent prosthesis 100.

FIG. 4A illustrates an unassembled configuration of the stent frame 400. FIG. 4B illustrates a side view of the stent frame 400 in an assembled configuration forming a tubular stent frame. FIG. 4C is a perspective view of the stent frame 400 in an assembled configuration forming a tubular stent frame. In the illustrated embodiment, the stent frame 400 includes a frame 402 with a plurality of arms 404 forming apexes 406. The laterally adjacent arms 404 form V-shaped structures. Accordingly, the frame 402 forms a plurality a V-shaped structures. Vertically adjacent V-shape structures can form diamond shaped structures.

In the illustrated embodiment of the stent frame 400, the frame 402 comprises eight rows of arms 404. The rows of the frame 402 may be coupled together by struts 408. The struts 408 may couple vertically adjacent apexes 406. The length of the struts 408 between the bottom two rows and between the top two rows are shorter than the length of the struts 408 between the central rows. Further, the struts 408 between the bottom two rows and the top two rows are disposed between every other apex 406. The struts 408 disposed between the center rows are offset from the struts 408 between adjacent rows. The struts 408 in these rows may be separated by several apexes 406.

In some embodiments, the stent frame 400 may be cut from a sheet of material. For example, the stent frame 400 may be cut from a sheet a material and then formed into a tubular shape. For example, a first end 410 of the stent frame 400 may be coupled to a second end 412 of the stent frame 400 to form a tubular shape. In some embodiment, the stent frame 400 may be cut from a tube of material.

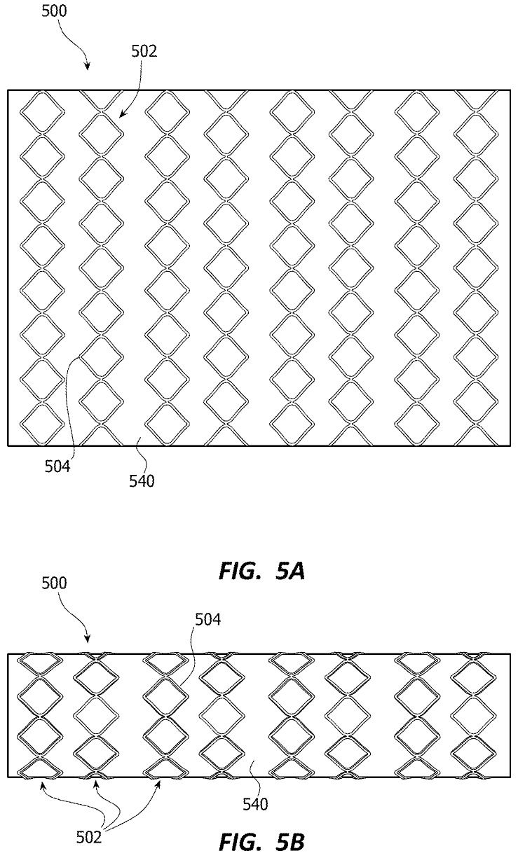

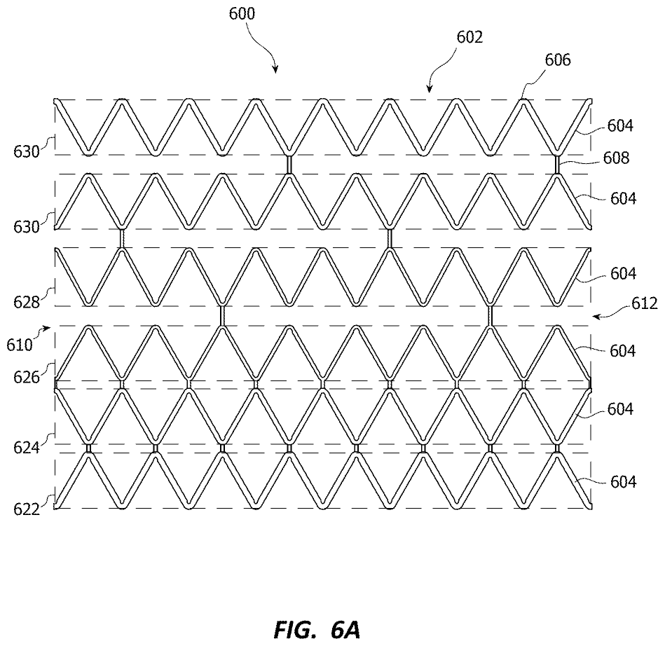

FIGS. 5A and 5B illustrate a stent frame 500 according to one example of the present disclosure. The stent frame 500 may be utilize in the balloon expandable branching stent prosthesis 200. For example, the stent frame 500 may be utilized as the stent frame 210, 220, 230 in the balloon expandable branching stent prosthesis 200.

FIG. 5A illustrates a side view of the stent frame 500 with a tubular shape. FIG. 5B is a perspective view of the stent frame with a tubular shape. In the illustrated embodiment, the stent frame 500 includes a plurality of annular rings 502. Each annular ring 502 has a plurality of diamonds 504. The plurality of annular rings 502 are coupled together by a cover 540. In a compressed configuration, the diamonds are flattened, and in an expanded configuration, the diamonds are elongated.

In some embodiments, the stent frame 500 may be cut from a sheet of material. For example, the stent frame 500 may be cut from a sheet a material and then formed into a tubular shape. For example, a first end of the stent frame 500 may be coupled to a second end of the stent frame 500 to form a tubular shape. In some embodiment, the stent frame 500 may be cut from a tube of material.

The stent frames 300, 400, 500 discussed above may be expanded from an undeployed/compressed configuration to an expanded configuration by one or more balloons. For example, a balloon may be inserted into the lumen of the stent frame 300, 400, 500, and the balloon may be expanded with a fluid (e.g., air, water, saline, and the like) which expands the stent frame 300, 400, 500.

During inflation of the balloon and expansion of the stent frame 300, 400, 500, the balloon may begin to dog-bone. Dog-boning is a term of art referring to the expansion of balloon (e.g., angioplasty balloon) at the proximal and distal ends—likened to a dog bone—before the expansion of the center of the balloon. Dog-boning can cause the stent frame to expand inconsistently, which may cause the stent frame 300, 400, 500 itself to bone done. In an effort to control the expansion of the stent frame, the individuals arms 304, 404 or annular rings 502 of the stent frame may have different thicknesses.

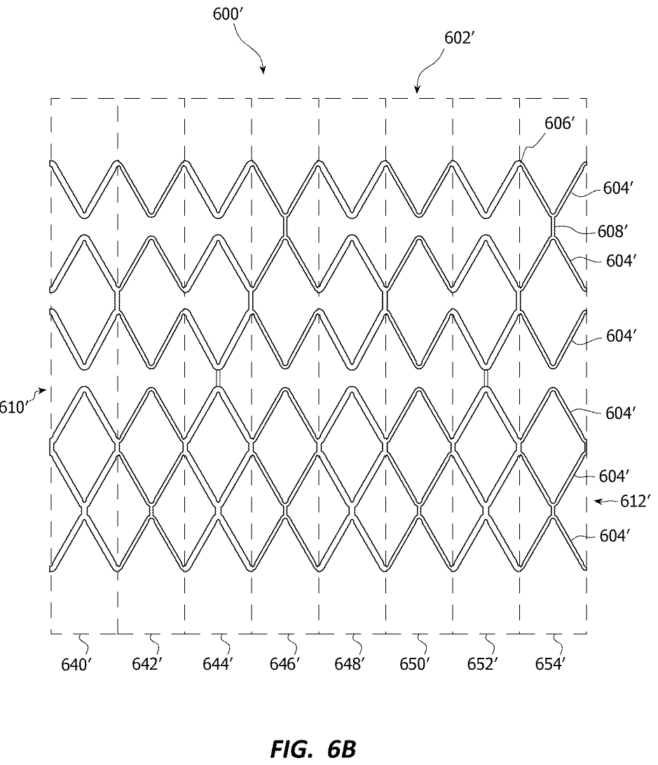

FIGS. 6A and 6B illustrate a stent frame 600, 600′ similar to the stent frame 300 of FIG. 3A. However, the present disclosure is not so limited and the description of FIGS. 6A and 6B may be easily applied to other stent frame structures, such as the stent frame 400 of FIG. 4A-4C and the stent frame 500 of FIGS. 5A-5B. The stent frame 600, 600′ includes a frame 602, 602′ with a plurality of arms 604, 604′ forming apexes 606, 606′. The laterally adjacent arms 604, 604′ form V-shaped structures. Accordingly, the frame 602, 602′ forms a plurality a V-shaped structures. Vertically adjacent V-shape structures of the frame 602, 602′ can form diamond shaped structures. For ease of discussion, the stent frame 600, 600′ is shown in a flat or unassembled configuration rather than in a tubular shape, however, the design of the stent frame 600, 600′ is to better control the expansion of the stent frame 600, 600′ from a compressed tubular shape to an expanded tubular shape. To assemble the stent frame into a tubular shape, a first end 610, 610′ is coupled to a second end 612, 612′, thereby forming a tubular shape.

FIG. 6A illustrates an embodiment of the stent frame 600 with a plurality of arm thickness zones 620, 622, 624, 626, 628, and 630. For description purposes, thickness zone 620 is at a proximal end of the stent frame 600 and thickness zone 630 is at a distal end of the stent frame 600. The thickness of arms 604 of zones 624, 626 may be the same. The thickness of arms 604 of zones 622, 628 may be the same and may be thicker than the arms 604 of zones 624, 626. The thickness of arms 604 in zones 620, 630 may be the same and the may be thicker than the arms 604 of zones 622, 628, and thicker than the arms 604 of zones 624, 626. In other words, the thickness of the arms 604 of the stent frame 600 are thinnest in the center of the stent frame 600 and the arms 604 gradually thicken as the stent frame 600 extends toward the distal and proximal ends of the stent frame 600. Accordingly, the thickness of the arms 604 change along the longitudinal length (e.g. the illustrated vertical length) of the stent frame 600.

The differences in the thicknesses of the arms 604 enables the stent frame to expand consistently in a predetermined order. For example, as the balloon expands, the thinnest arms 604 of zones 624, 626 expand first because it takes the least amount of force to expand the thinnest arms 604 in zones 624, 626. Since the arms 604 of zones 620, 630 are thickest they counter the dog-boning of the balloon, enabling the thinner arms 604 of zones 624, 626 to expand first. The arms 604 of zones 622, 628 expand next as they are thinner than the arms 604 of zone 620, 630, but are thicker than the arms 604 of zones 624, 626. The arms of zones 620, 630 expand last as they are the thickest of the arms 604.

As the arms 604 of each zone expand, the arms 604 are strain harden so that they arms 604 get stiffer and the arms 604 in subsequent zones may be expanded. Accordingly, the stent frame 600 avoids dog-boning because the central arms 604 expand before the arms 604 at the proximal end (e.g., zone 620) and the arms 604 at the distal end (e.g., zone 630).

The difference in thicknesses of the arms 604 between zones may range between 0.5 to 10 percent. This difference in thicknesses are greater than tolerances that occur during CNC (Computerized Numerical Control) machining. For example, the difference in thickness between zones 624, 626 and zones 622, and 628 may be around 3 percent. The different in thickness between zones 624, 626, and zones 620, 630 may be around 6 percent or double the thickness difference between the zones 624, 626, and zones 622, 628.

In some embodiments, the longitudinal thickness zones may extend beyond a single stent frame. For example, the balloon expandable branching stent prosthesis 100 may comprise longitudinal thickness zones that extend from the first stent frame 110 to the second stent frame 120 and the third stent frame 130, so that the proximal end of the first stent frame 110 and distal ends of the second stent frame 120 and the third stent frame 130 are thicker than a distal end of the first stent frame 110 and proximal ends of the second stent frame 120 and the third stent frame 130. In other words, the thickness of the arms in the center of the balloon expandable branching stent prosthesis 100 are thinner near the crotch portion 108 than at the free ends of the trunk portion 102, the first branching portion 104, and the second branching portion 106. This helps prevent dog-boning of the balloon expandable branching stent prosthesis 100.

FIG. 6B illustrates an embodiment of the stent frame 600′ with a different set of a plurality of arm thickness zones 640′, 642′ 644′, 646′, 648′, 650′, 652′, 654′ that extend from the first end 610′ to the second end 612′. The thickness of the arms 604′ may alternate from the first end 610′ to the second end 612′. The thickness of arms 604 in zones 640′, 644′, 648, 652′ may be the same and the thickness of the arms 604 in zones 642′, 646′, 650′, 654′ may be the same. In the illustrated embodiment, the thickness of arms 604 in zones 642′, 646′, 650′, 654′ are thicker than the arms 604′ in zones 640′, 644′, 648′, 652′. Accordingly, the thickness of the arms 604′ change along the circumference of the stent frame 600′.

The differences in the thicknesses of the arms 604′ enables the stent frame 600′ to expand consistently in a predetermined order. For example, as the balloon expands, the thinnest arms 604′ of zones 640′, 644′, 648′, 652′ expand first because it takes the least amount of force to expand the thinnest arms 604′ in zones 640′, 644′, 648′, 652′. The arms 604 of zones 642′, 646′, 650′, 654′ expand next as they are thicker than the arms 604′ of zones 640′, 644′, 648′, 652′.

As the arms 604′ of each zone expand, the arms 604′ are strain harden so that they arms 604′ get stiffer and the arms 604′ in subsequent zones may be expanded.

The difference in thicknesses of the arms 604′ between zones may range between 0.5 to 10 percent. This difference in thicknesses are greater than tolerances that occur during CNC machining. For example, the difference in thickness between zones 642′, 646′, 650′, 654′ and zones 640′, 644′, 648′, 652′ may be around 3 percent.

In some embodiments, there may be arm thickness zones with three or more distinct thicknesses that alternate along the circumference of the stent frame.

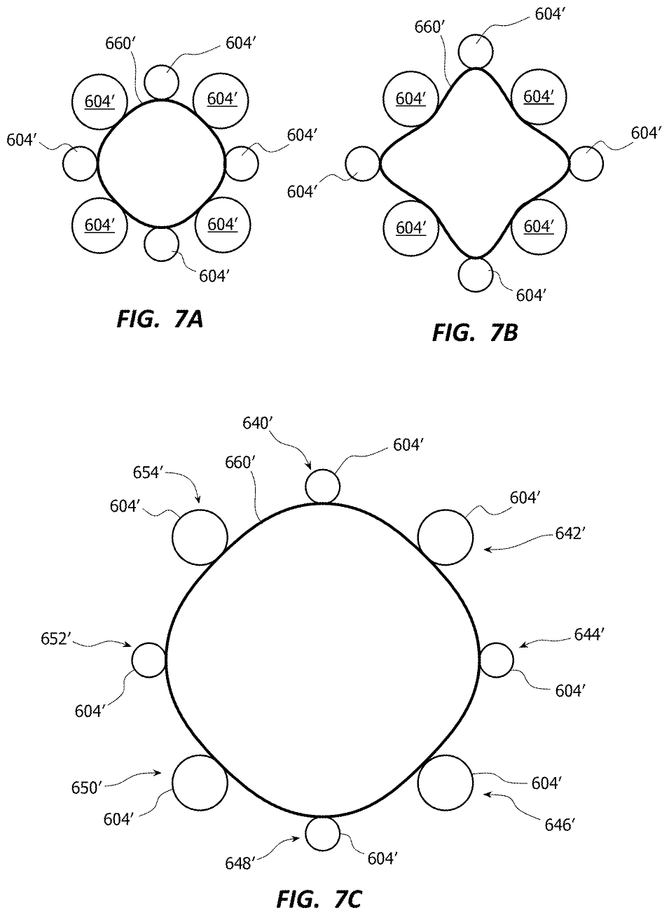

FIGS. 7A-7C illustrate the process of expanding the stent frame 600′ and a cover 660′ of the stent frame 600′. FIG. 7A is a cross-sectional view of the stent frame 600′ in a compressed configuration. In the illustrated embodiment, arms 604′ are circumferentially spaced apart around a circumference of the stent frame 600′. As discussed above, the thickness of the arms 604′ alternate between varying thickness around the circumference of the stent frame 600′. The illustrated embodiment of the stent frame 600′ includes eight different thickness zones that alternate around the circumference of the stent frame 600′ and change about every 45 degree about the circumference. The thickness zone 640′ is disposed at the top of the stent frame 600′, the thickness zone 644′ is disposed at the right of the stent frame 600′, the thickness zone 648′ is disposed at the bottom of the stent frame 600′, and the thickness zone 652′ is disposed at the left of the stent frame 600′, all of which have the same arm 604′ thickness.

The thickness zone 642′ is disposed between the thickness zone 640′ and thickness zone 644′, the thickness zone 646′ is disposed between the thickness zone 644′ and 648′, the thickness zone 650′ is disposed between the thickness zone 648′ and the thickness zone 652′, and thickness zone 654′ is disposed between the thickness zone 652′ and 640′, all of which have the same arm 604′ thickness which is greater than the arm 604′ thickness of arm 604′ thickness of the thickness zones 640′, 644′, 648′, 652′.

FIG. 7B illustrates the stent frame 600′ in a partially expanded configuration. The stent frame 600′ may be expanded by a balloon (not shown). As the balloon is expanded, the balloon provides a radially outward force to the stent frame 600′. As discussed above, the arms 604′ in thickness zones 640′, 644′, 648′, 652′ expand first because of the thinner arm 604′ thickness. FIG. 7B illustrates the arms 604′ of thickness zones 640′, 644′, 648′, 652′ are expanded radially outward while the arms 604′ of thickness zones 642′, 646′, 650′, 654′ are maintained in their compressed configuration. The arms 604′ of thickness zones 640′, 644′, 648′, 652′ become strain hardened due to their expansion by the balloon.

FIG. 7C illustrates the stent frame 600′ in a fully expanded configuration. The stent frame 600′ is further expanded by the balloon (not shown). As the balloon is further expanded, the balloon provides a radially outward force to the stent frame 600′. As discussed above, the arm 604′ in thickness zones 642′, 646′, 650′, 654′ expand next because the thicker arm 604′ thickness. FIG. 7C illustrates the arms 604′ of all of the thickness zones 640′, 642′, 644′, 646′, 648′, 650′, 652′, 654′ are at an expanded configuration. The arms 604′ of thickness zones 642′, 646′, 650′, 654′ become strain hardened due to their expansion by the balloon. Accordingly, the expansion of the stent frame 600′ is controlled by the varying thickness of the various arms 604′ of the stent frame 600′.

The present disclosure is not limited to the above-disclose thickness zones and other types of thickness zone designs are within the scope of the present disclosure. For example, in some embodiments, the stent frame may also include thickness zones that vary helically about the stent frame. In some embodiments, the thickness zones may alternate about the circumference of row of the stent frame, and thickness zones of various rows of the stent frame may be offset from each other. In some embodiments, the thickness zones may embody the thickness zones of FIGS. 6A and 6B together. In other words, the stent frame may include thickness zones in the longitudinal direction of the stent frame and may include circumferential thickness zones that work together in combination.

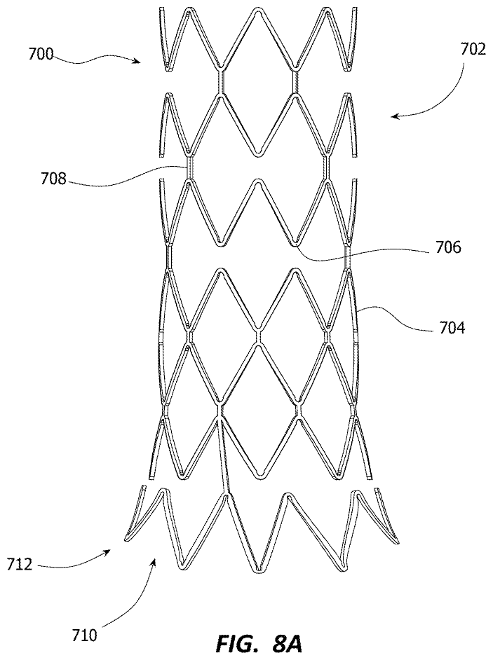

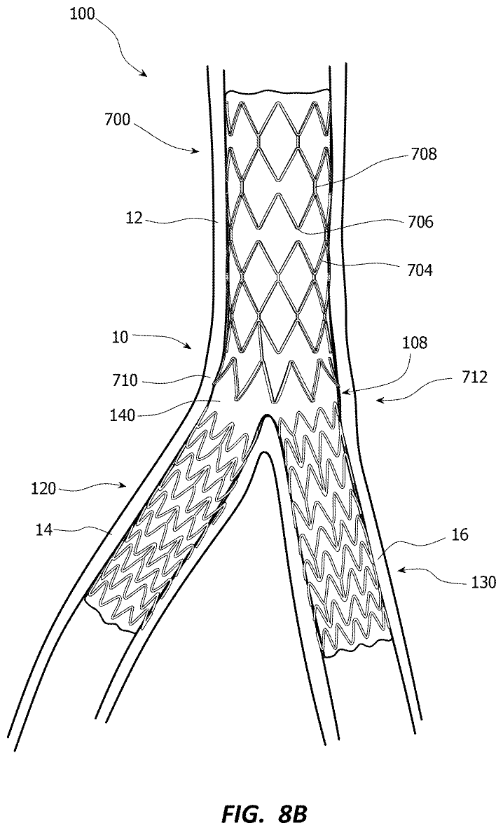

FIG. 8A illustrates a side view of an embodiment of a stent frame 700 according to one embodiment of the present disclosure. The stent frame 700 includes a frame 702 with a plurality of arms 704 forming apexes 706. The laterally adjacent arms 704 form V-shaped structures. Accordingly, the frame 702 forms a plurality a V-shaped structures. Vertically adjacent V-shape structures can form diamond shaped structures.

The stent frame 702 comprises multiple rows of arms 704. The rows of the frame 702 may be coupled together by struts 708. The struts 708 may couple vertically adjacent apexes 706. The length of the struts 708 may vary between struts 708 connected various rows together.

A distal end 710 of the frame 702 may comprise a flared end 712 that flares radially outward. For example, a row of arms 704 at the distal end 710 of the frame 720 comprises arms 704 that flare outward. The stent frame 700 may be external to a cover, internal to a cover, or disposed within the cover.

FIG. 8B illustrates a deployed balloon expandable branching stent prosthesis 100 with the stent frame 700 utilized in the trunk portion 102 of the balloon expandable branching stent prosthesis 100 at the branching implant site 10 within the anatomical system in which it is deployed. The flared end 712 is disposed at the bifurcation of the deployed balloon expandable branching stent prosthesis 100.

Because of the spacing between the stent frame 110, 120, 130 of the balloon expandable stent prosthesis 100, when the balloon expandable stent prosthesis 100 is compressed, the cover 140 may radially infold beyond the stent frames 110, 120, 130 and thus minimizing the lumen of the balloon expandable stent prosthesis 100. This can cause problems with flow. Accordingly, the crotch portion 108 portion of the balloon expandable stent prosthesis can be reinforced to prevent radial infolding of the cover 140. The flared end 712 of the stent frame 700 is designed reinforces for the crotch portion 108 of the balloon expandable branching stent prosthesis 100 in comparison to the balloon expandable branching stent prosthesis 100 of FIG. 1A to prevent radial infolding the cover 140.

The flared end 712 of the stent frame 700 is flared during the manufacturing process and the flared end 712 does not occur simply because the expansion of the stent frame 700. In some embodiments, the stent frame 700 is fabricated from a non-superelastic material (e.g. memory material). The stent frame 700 may be manufactured from a number of metals or materials that have low yield stress and high elastic modulus. Low yield and high elastic modulus enable the stent frame 700 to the shape needed, make it deformable at manageable balloon pressures, and have minimal recoil when the balloon is deployed. Exemplary materials include stainless steel, tantalum, tantalum alloys, platinum alloys, niobium alloys, cobalt alloys, other metallic alloys, polymers, and the like. In some embodiments, the stent frame 700 is fabricated on a flared mandrel to form the flared end 712 of the stent frame 700.

In some embodiments, the flared end 712 of the stent frame 700 may have a non-circular cross-section. In some embodiments, the flared end 712 of the stent frame 700 may have an oval cross-section. The non-circular cross-section of the flared end 712 of the stent frame 700 causes the stent frame 700 is lose some flexibility compared to the rest of the stent frame 700. While the flared end 712 loses some flexibility compared to the rest of the stent frame 700, the non-circular cross-section reinforces the crotch portion 108 of the balloon expandable branching stent prosthesis 100.

In the illustrated deployed configuration, the trunk portion 102 and the stent frame 700 of the balloon expandable branching stent prosthesis 100 is deployed at/within the trunk 12 of the branching implant site 10, the first branching portion 104 and the stent frame 120 of the balloon expandable branching stent prosthesis 100 is deployed at/within the first branch vessel 14 of the branching implant site 10, and the second branching portion 106 and the stent frame 130 of the balloon expandable branching stent prosthesis 100 is deployed at/within the second branch vessel 16 of the branching implant site 10.

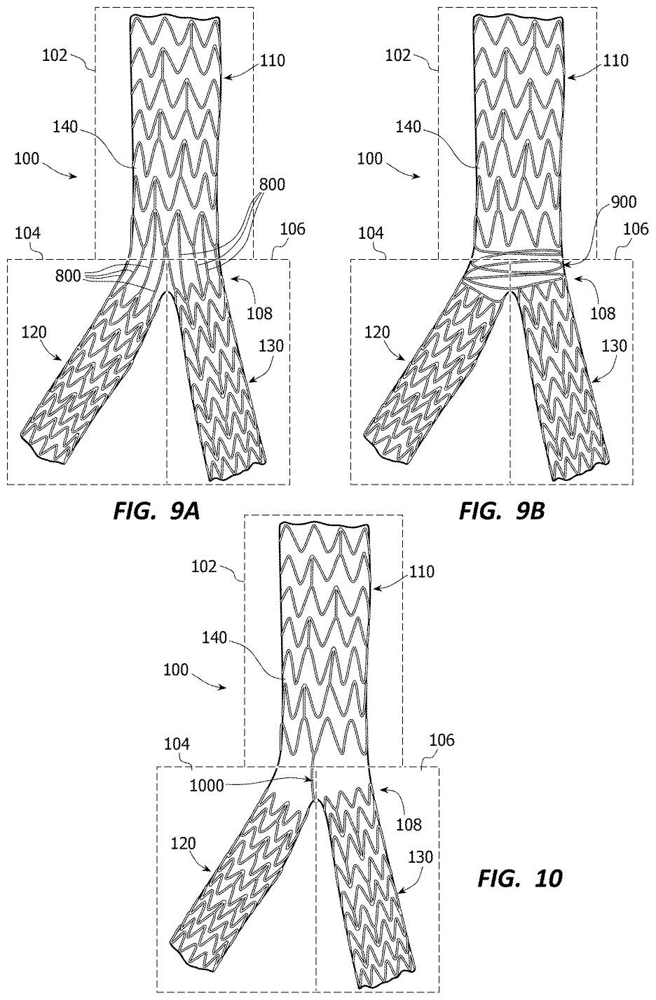

The crotch portion 108 of the balloon expandable branching stent prosthesis 100 may be strengthened in a number of different ways. FIG. 9A illustrates the balloon expandable branching stent prosthesis 100 with the trunk portion 102, the first branching portion 104, the second branching portion 106 and the cover 140. The trunk portion 102 is fortified by stent frame 110, the first branching portion 104 is strength by the stent frame 120, and the second branching portion 106 is strengthen by the stent frame 130.

The crotch portion 108 may be strengthened with a plurality of longitudinal beadings 800 of a reinforcing material. The beadings 800 may extend from the trunk portion 102 of the balloon expandable branching stent prosthesis 100 to the first branching portion 104 or to the second branching portion 106. Accordingly, the crotch portion 108 is fortified even without a stent frame located in the crotch portion 108. The beadings 800 may be external to the cover 140, internal to the cover 140, or disposed within the cover 140. The beadings 800 are fabricated of a suitable material that may reinforce the crotch portion 108. For example, a suitable material for the beadings may be FEP, polytetrafluoroethylene (PTFE), and/or other materials.

FIG. 9B illustrates another embodiment of fortifying the crotch portion 108 of the balloon expandable branching stent prosthesis 100. The crotch portion 108 may be strengthened with a one or more helical beadings 900 of a reinforcing material. The beading 900 may helically extend from the trunk portion 102 of the balloon expandable branching stent prosthesis 100 to the first branching portion 104 and/or the second branching portion 106. Accordingly, the crotch portion 108 is fortified even without a stent frame located in the crotch portion 108. The beadings 900 may be external to the cover 140, internal to the cover 140, or disposed within the cover 140. The beadings 900 are fabricated of a suitable reinforcing material that may reinforce the crotch portion 108. For example, the reinforcing material may be FEP, PTFE, and/or other materials.

FIG. 10 illustrates another embodiment of fortifying the crotch portion 108 of the balloon expandable branching stent prosthesis 100. The crotch portion 108 may be strengthened with a strap 1000 that extends from a first side of the stent frame 110 around the crotch portion 108 between the first branching portion 104 and the second branching portion 106 and toward a second side of the stent frame 110. The strap 1000 may be external to the cover 140, internal to the cover 140, or disposed within the cover 140.

In some embodiments, the crotch portion 108 of the cover 140 may be a composite material to reinforce the crotch portion 108. For example, in some embodiments, the crotch portion 108 may comprise a Kevlar composite material.

In some embodiments, the balloon expandable branching stent prosthesis 100 may be dilated past a nominal diameter of the balloon expandable branching stent prosthesis 100. A nominal diameter is the diameter of the balloon expandable branching stent prosthesis 100 is designed to expand during inflation. In some situations, the balloon expandable branching stent prosthesis 100 may be expanded or dilated past the nominal diameter of the expandable stent prosthesis. Specifically, in some situations and in some embodiments, the balloon expandable branching stent prosthesis 100 may be serially dilated so that the balloon expandable branching stent prosthesis 100 expands past the nominal diameter of the balloon expandable branching stent prosthesis 100. This may be useful in the treatment of the implant site due to diseased, misshapen, and/or damaged vessels. Serially dilation of the balloon expandable branching stent prosthesis 100 may be accomplished through the use of gradually larger diameter balloons to enable the balloon expandable branching stent prosthesis 100 to expand past its nominal diameter.

As discussed above, in embodiments where the cover 140 comprises the inner layer, the outer layer, and the tie layer that is configured to be a tissue impermeable layer. Thus, the tie layer may be configured to create a mid-layer portion of a construct and the tie layer is tissue impermeable to inhibit tissue or cellular ingrowth into the layer or to be impervious to tissue migration into or through the layer or to substantially inhibit tissue migration.

Accordingly, when the balloon expandable branching stent prosthesis 100 is expanded beyond its nominal diameter, it is important that the cover 140 maintain the tissue impermeability of the tie layer. Tears or rips in the tie layer, even microtears, can affect the tissue impermeability of the tie layer and enable tissue or biological fluid to migrate through the tie layer of the cover 140. Accordingly, the tie layer of the cover 140 may be designed so that the tie layer may expand beyond the nominal diameter of the balloon expandable branching stent prosthesis 100 without comprising the tissue impermeability of the tie layer.

In some embodiments, the balloon expandable branching stent prosthesis 100 may be manufactured so that the tie layer of the cover maintains its tissue impermeability up to specific ratio of expansion or dilation beyond the nominal diameter. In some embodiments, the tie layer of the cover 140 maintain its tissue impermeability up to a fifty percent expansion of the balloon expandable branching stent prosthesis 100 beyond its nominal diameter. Embodiment wherein the cover 140 maintain its tissue impermeability up to a fifty percent expansion, forty percent expansion, thirty percent expansion, twenty percent expansion, or embodiments wherein the cover 140 maintains its tissue impermeability greater than twenty percent, greater than thirty percent, or greater than forty percent of the balloon expandable branching stent prosthesis 100 beyond its nominal diameter are all with the scope of this disclosure. In some embodiments, the tie layer of the cover 140 maintains its tissue impermeability up to 6 mm change in diameter relative to the nominal diameter of the balloon expandable branching stent prosthesis 100. While, the tissue impermeability of the tie layer is described above in relation to the balloon expandable branching stent prosthesis 100, the methods of manufacturing described below are in relation to a tubular shaped stent prosthesis and manufacturing process may be applied to the balloon expandable branching stent prosthesis 100.

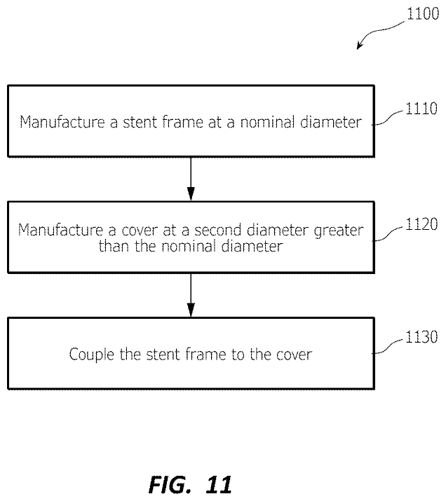

FIG. 11 illustrates a flowchart 1100 for a process to manufacture a stent prosthesis that may be expanded beyond its nominal diameter and maintain the tissue impermeability of the cover when the stent prosthesis is expanded up to fifty percent beyond its nominal diameter or up to 6 mm beyond its nominal diameter. As also noted above, other degrees of expansion beyond the nominal diameter, including forty percent, thirty percent, twenty percent, greater than twenty percent, greater than thirty percent, or greater than forty percent are all within the scope of this disclosure.

Step 1110 comprises manufacturing a stent frame for a tubular prosthesis at a nominal diameter. In some embodiments, the stent frame may be fabricated from a extruded piece of a stainless steel, tantalum, tantalum alloys, platinum alloys, niobium alloys, cobalt alloys, other metallic alloys, polymers, and the like. The stent frame may be fabricated with laser cutting on a CNC machine to form the stent frame. The stent frame may have one of the stent frame structures discussed above in relation to FIGS. 3A-3C, 4A-4C, and 5A-5C. As discussed above, the stent frame may be cut with the above described varying thickness zones for the arms of the stent for controlling the expansion of the stent in a predetermined order. The extruded piece of material may have a tubular shape and diameter and diameter of the tubular shape may be a nominal diameter for the stent frame. In some embodiments, the tubular extrusion and may extruded on a mandrel. Once cut, the stent frame is configured to be compressed to a compressed configuration.

In some embodiments, the stent frame is laser cut using a CNC machine on a flat piece of material, such as stainless steel, tantalum, tantalum alloys, platinum alloys, niobium alloys, cobalt alloys, other metallic alloys, polymers, and the like. The flat piece of material may be formed into a tubular shape on a mandrel that defines the nominal diameter of the stent frame. The lateral edges of the flat piece of material may be coupled together to form a tubular stent frame. In some embodiments, the stent frame may be a braided stent frame that is braided on a mandrel at the nominal diameter.

Step 1120 comprises manufacturing a cover for the stent prosthesis. The cover for the stent prosthesis is fabricated on a mandrel that is greater than the nominal diameter of the stent frame. The diameter of the mandrel may be at least fifty percent greater than the nominal diameter of the stent frame or at least 6 mm greater than the nominal diameter of the stent frame. Additionally, embodiment wherein the diameter of the mandrel may be at least forty percent greater, thirty percent greater, twenty percent greater, more than twenty percent greater, more than thirty percent greater, or more than forty percent greater than the nominal diameter of the stent frame are all within the scope of this disclosure. As discussed above, the cover of the stent prosthesis may comprise an inner layer, and outer layer, and a tie layer. The cover may be applied to the mandrel in a number of different ways, such as electrospinning the cover onto the mandrel, rotational spinning the cover onto the mandrel, extruding the cover onto the mandrel. In some embodiments, the fibril alignment of the cover may be in a hoop direction instead of a longitudinal direction to permit expansion of the cover in the radial direction.

Step 1130 comprises coupling the cover to the stent prosthesis. In some embodiments, step 1120 and 1130 are simultaneous as the cover is coupled to the stent frame as the cover is manufactured. For example, the stent frame may be placed on the mandrel that is greater than the nominal diameter of the stent frame and the cover in applied to the mandrel and the stent frame.

Accordingly, when the stent prosthesis is implanted in a patient's vasculature, the stent may be expanded to the nominal diameter. In some situations, the stent prosthesis may be expanded beyond its nominal diameter using a greater diameter balloon that is greater than the nominal diameter of the stent frame. When the stent prosthesis is expanded beyond its nominal diameter, the tissue impermeability of the tie layer of the cover is maintain up to fifty percent greater than the nominal diameter of the stent frame.

In other embodiments, the stent frame is coupled to the stent frame after the cover is manufactured. For example, the cover is manufactured on the mandrel and then the stent frame is coupled to the cover on a luminal surface of the cover or on the abluminal surface of the cover and the cover is manufactured.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

1. A balloon expandable branching stent prosthesis comprising:
a trunk portion including a trunk portion stent frame;
a first branching portion including a first branching portion stent frame;
a second branching portion including a second branching portion stent frame;
a cover coupling the trunk portion stent frame, the first branching stent frame, and the second branching stent frame together forming a bifurcated stent, wherein the trunk portion stent frame, the first branching portion stent frame, and the second branching portion stent frame are spaced apart from each other with the cover disposed therebetween; and
a lumen that extends through the trunk portion, the first branching portion, and the second branching portion,
wherein a crotch portion of the balloon expandable branching stent prosthesis is disposed between the trunk portion, the first branching portion, and the second branching portion,
wherein the balloon expandable branching stent prosthesis is expandable by an inflatable balloon disposed within the lumen of the expandable branching stent prosthesis, and
wherein the crotch portion is reinforced to prevent radial infolding of the cover.

2. The balloon expandable branching stent prosthesis of claim 1, wherein the trunk portion stent frame comprises a flared end that flares radially outward in the crotch portion to reinforce the crotch portion of the balloon expandable branching stent prosthesis.

3. The balloon expandable branching stent prosthesis of claim 2, wherein the trunk stent frame is fabricated from a non-superelastic material.

4. The balloon expandable branching stent prosthesis of claim 2, wherein the flared end of the trunk stent frame comprises an oval cross-section.

5. The balloon expandable branching stent prosthesis of claim 1, further comprising a longitudinal beading of a reinforcing material that extends from the trunk portion into the first branching portion through the crotch portion and from the trunk portion into the second branching portion through the crotch portion.

6. The balloon expandable branching stent prosthesis of claim 5, wherein the reinforcing material is fluorinated ethylene propylene (FEP) or polytetrafluoroethylene (PTFE).

7. The balloon expandable branching stent prosthesis of claim 1, wherein the crotch portion of the balloon expandable branching stent prosthesis comprises a composite material to reinforce the crotch portion.

8. The balloon expandable branching stent prosthesis of claim 1, further comprising a strap that that extends from a first side of the trunk portion stent frame between the first branching portion and the second branching portion to a second side of the trunk portion stent frame opposite the first side.

9. The balloon expandable branching stent prosthesis of claim 1, wherein each stent frame comprises a plurality of arms that form a plurality of apexes, wherein a thickness of the arms vary and are designed to be expanded by the balloon in a predetermined order based on the thickness of the arms.

10. The balloon expandable branching stent prosthesis of claim 9, wherein the plurality of arms comprises a plurality of thickness zones, wherein each thickness zone comprises a predetermined thickness.

11. The balloon expandable branching stent prosthesis of claim 10, wherein the plurality of thickness zones are arranged longitudinally, such that an arm thickness of the arms in the thickness zones of a central portion of each stent frame are thinner than an arm thickness of the arms in the thickness zones of a distal portion and a proximal portion of each stent frame.

12. The balloon expandable branching stent prosthesis of claim 1, wherein the cover comprises a tissue impermeability layer, and
wherein the tissue impermeability layer maintains tissue impermeability when the cover is expanded beyond a nominal diameter.

13. A stent frame for a balloon expandable stent comprising:
a plurality of arms that form a plurality of apexes, wherein a thickness of the arms vary and are designed to be expanded by a balloon in a predetermined order based on the thickness of the arms.

14. The stent frame of claim 13, wherein the plurality of arms are formed in a plurality of circumferential rows, wherein apexes of adjacent rows are coupled together by a plurality of struts.

15. The stent frame of claim 13, wherein the plurality of arms are formed in a plurality of circumferential rings that are longitudinally spaced apart along a length of the stent frame, and
wherein the plurality of circumferential rings are coupled together by a cover.

16. The stent frame of claim 13, wherein the plurality of arms comprises a plurality of thickness zones, wherein each thickness zone comprises a predetermined thickness.

17. A method of manufacturing a stent, comprising:
manufacturing a stent frame for the stent at a nominal diameter;
manufacturing a cover for the stent at a diameter that is greater than the nominal diameter of the stent frame; and
coupling the stent frame to the cover.

18. The method of claim 17, wherein manufacturing the stent frame comprises laser cutting the stent frame.

19. The method of claim 17, wherein the cover comprises a tie layer that is tissue impermeable.

20. The method of claim 17, wherein the stent expands to a diameter forty percent greater than the nominal diameter and the tie layer remains tissue impermeable.
